(19) United States
(12) Patent Application Publication
(10) Pub. No.: US 2025/0387126 A1
(43) Pub. Date: Dec. 25, 2025

(54) Resection Guide And Flex Plate To Alter Distal Femoral Flexion

(71) Applicant: Howmedica Osteonics Corp., Mahwah, NJ (US)

(72) Inventors: Kerianne Coulon, Hoboken, NJ (US); Sophie Hatcher, Weehawken, NJ (US)

(21) Appl. No.: 19/247,350

(22) Filed: Jun. 24, 2025

Related U.S. Application Data
(60) Provisional application No. 63/663,726, filed on Jun. 25, 2024.

(51) Int. Cl. A61B 17/15

(52) U.S. Cl. CPC A61B 17/155

(57) ABSTRACT

A cutting guide is provided for resecting a bone to receive an implant. The cutting guide includes a body configured to be positioned adjacent to the bone and a slot extending through the body and configured to receive a cutting edge for resecting the bone. The cutting guide further comprises a first hole extending though the body and configured to receive a pin anchored to the bone, the first hole being configured to position the slot along a first plane when the first hole is passed over the pin. The cutting guide further comprises a second hole extending though the body and configured to receive the pin anchored to the bone, the second hole being configured to position the slot along a second plane when the second hole is passed over the pin, wherein first plane is transverse to the second plane.

### CROSS-REFERENCE TO RELATED APPLICATIONS

This application claims the benefit of the filing date of U.S. Provisional Patent Application No. 63/663,726 filed Jun. 25, 2024, the disclosure of which is hereby incorporated herein by reference in its entirety.

### BACKGROUND OF THE INVENTION

Revision total knee arthroplasty (TKA) procedures involve the replacement of portions of the patella, femur and tibia with artificial components. In particular, at least part of a procedure may involve resection of a distal portion of the femur to be replaced with artificial components. There are several types of knee prostheses known in the art. One type is sometimes referred to as a “resurfacing type”. In these prostheses, the articular surface of the distal femur is “resurfaced” with respective metal and plastic condylar-type articular bearing components. These knee prostheses provide adequate rotational and translational freedom and require minimal bone resection to accommodate the components within the boundaries of the available joint space. Additionally, the femoral components are usually provided with an intramedullary (IM) stem option.

However, those skilled in the art will appreciate that revision TKA procedures are difficult because (1) the type and location of cavernous defects make it difficult to match the exterior surfaces of the tibia and femur to the interior surfaces of the prosthesis, (2) the femur and tibia must be resected with reference to the IM canal, and (3) the use of multiple templates and guides during the course of the procedure makes it very difficult to keep all the cuts in proper alignment relative to the IM canal.

Additionally, during revision TKA procedures, it is desirable to balance the knee in both flexion and extension. For example, the femur is typically positioned in a flexion relative to a mechanical axis in order to reduce the flexion gap and support a more well-balanced knee. However, there lacks a consistent and predictable approach for controlling the amount of flexion added prior to resecting the distal femur. Further, there also lacks an approach for determining whether the added amount of flexion properly reduces the flexion gap prior to resecting the distal femur.

Accordingly, a need exists for improved instrumentation and methods for preparation of a long bone for receipt of an implant.

### BRIEF SUMMARY OF THE INVENTION

In a first example of a first aspect, the present disclosure relates to a cutting guide for resecting a bone to receive an implant. The cutting guide comprises a body configured to be positioned adjacent to the bone and a slot extending through the body and configured to receive a cutting edge for resecting the bone. The cutting guide further comprises a first hole extending though the body and configured to receive a pin anchored to the bone, the first hole being configured to position the slot along a first plane when the first hole is passed over the pin. The cutting guide further comprises a second hole extending though the body and configured to receive the pin anchored to the bone, the second hole being configured to position the slot along a second plane when the second hole is passed over the pin. The first plane and the second plane are both orthogonal to a third plane, and the first plane is transverse to the second plane, and the cutting guide is configured such that when the body is received on the pin anchored to the bone, the first plane represents a first path of resection relative to the bone and the second plane represents a second path of resection relative to the bone.

In a second example, the first example of the first aspect is further defined wherein the body is configured to be positioned adjacent an anterior side of the bone such that the cutting edge cuts in an anterior-posterior direction. In a third example, the first example of the first aspect is further defined wherein an angle between the first plane and the second plane is one of 2 degrees, 4 degrees or 6 degrees. In a fourth example, the first example of the first aspect further comprises a third hole and a fourth hole, the third hole extending through the body and being configured to receive a second pin anchored to the bone when the first hole receives the pin, and the fourth hole extending through the body and being configured to receive the second pin anchored to the bone when the second hole receives the pin. In a fifth example, the fourth example of the first aspect is further defined wherein a longitudinal axis divides the body into a first and second side, the on the first hole is adjacent to the second hole on the first side, and the third hole is adjacent to the fourth hole on the second side.

In a sixth example, the first example of the first aspect is further defined wherein the second hole is non-parallel to the first hole. In a seventh example, the first example of the first aspect is further defined wherein the slot is proximate a first end of body and the first and second hole are proximate a second end of body opposite the first end, wherein the first end of body is configured to be proximate a first end of bone. In an eight example, the first example of the first aspect further comprises a support arm assembly configured to connect the guide to a reamer, wherein the reamer is configured to engage an intramedullary canal of the bone. In a ninth example, the eight example of the first aspect is further defined wherein the support arm assembly is further configured to position the slot along the first plane. In a tenth example, the first example of the first aspect further comprises a plate configured to be received by the slot, wherein the plate comprises an opening configured to receive a stem along the third plane. In an eleventh example, the first example of the first aspect further comprises a second slot extending through the body, wherein the second slot is positioned one of 5 millimeters, 10 millimeters, or 15 millimeters from the first slot.

In a first example of a second aspect, the present disclosure relates to a system for preparing a bone for receiving an implant. The system comprises a cutting guide configured to be attached to a bone, the cutting guide including a cutting slot and a plurality of holes, each hole of the plurality of holes being sized to receive a bone anchorage pin, wherein a first hole of the plurality of holes is configured to be received over a pin anchored to the bone so that the cutting guide is at a first orientation relative to the bone and a second hole of the plurality of holes is configured to be received over the pin anchored to the bone so that the cutting guide is at a second orientation relative to the bone. The system further comprises a plate releasably engaged to the slot of the cutting guide, the plate including an arm extending from the slot and a guide portion parallel to the slot, the guide portion including an opening therethrough. When the plate is engaged to the slot and the cutting guide is attached to the bone in the first orientation, the opening is aligned along a first central longitudinal axis and when the plate is engaged to the slot and the cutting guide is attached to the bone in the second orientation, the opening is aligned along a second central longitudinal axis, the first central longitudinal axis having a different anterior-posterior component than the second central longitudinal axis.

In a second example, the first example of the second aspect is further defined wherein the bone anchorage pin is fixed to the bone along a first axis extending along an anterior-posterior direction of the bone. In a third example, the first example of the second aspect further comprises a stem configured to engage the bone through the opening of the plate, wherein the bone is a left or right femur. In a fourth example, the third example of the second aspect is further defined wherein the opening of the guide portion includes a detent for retaining a portion of the stem. In a fifth example, the first example of the second aspect is further defined wherein an angle between the first central longitudinal axis and the second central longitudinal axis is one of 2 degrees, 4 degrees or 6 degrees. In a sixth example, the first example of the second aspect is further defined wherein each hole of the plurality of holes of the cutting guide are further configured to receive a second pin, wherein a third hole of the plurality of holes is configured to be received over the second pin anchored to the bone when the first hole is configured to be received over the pin anchored to the bone and a fourth hole of the plurality of holes is configured to be received over the second pin anchored to the bone when the second hole is configured to be received over the pin anchored to the bone.

In a first example of a third aspect, the present disclosure relates to a method for preparing a bone for receiving an implant. The method comprises positioning a cutting guide along a portion of a bone; anchoring a pin to the portion of the bone by passing the pin through a first hole in the cutting guide to align the cutting guide in a first orientation relative to the bone; removing the cutting guide from the pin; and advancing a second hole in the cutting guide over the pin anchored to the bone such that when the second hole receives the pin, the cutting guide is in a second orientation relative to the bone, wherein in the first orientation, a first plane passes through a slot of the cutting guide and in the second orientation, a second plane passes through the slot of the cutting guide, and wherein the first plane defines a cut line through the bone at a different slope in an anterior-posterior direction than the second plane.

In a second example, the first example of the third aspect further comprises prior to the positioning step, reaming an opening through the bone. In a third example, the second example of the third aspect is further defined wherein the opening is an intramedullary canal and the bone is a femur. In a fourth example, the second example of the third aspect is further defined wherein the positioning step further comprises configuring a support assembly to the cutting guide to position the cutting guide along the portion of the bone relative to the opening through the bone.

In a fifth example, the first example of the third aspect further comprises after the advancing step, engaging a plate with the cutting guide, wherein the plate is configured to receive a stem extending through an opening of the plate into an opening of the bone to define a trajectory of the stem. In a sixth example, the fifth example of the third aspect further comprises, after the engaging step, comparing a first trajectory of the stem in the first orientation of the cutting guide to a second trajectory of the stem in the second orientation of the cutting guide to determine a desired trajectory of the stem. In a seventh example, the sixth example of the third aspect further comprises, after the comparing step, removing the plate from the cutting guide. In an eight example, the seventh example of the third aspect further comprises, after the removing step, cutting a portion of the bone of the bone through the slot of the cutting guide when the cutting guide is in the first or second configuration that includes the desired trajectory of the stem.

### BRIEF DESCRIPTION OF THE DRAWINGS

The features, aspects, and advantages of the present disclosure will become better understood with regard to the following description, appended claims, and accompanying drawings in which:

FIG. 1 is a perspective view of a bone preparation system according to an embodiment of the present disclosure.

FIG. 2A is a front view of a resection guide of the bone preparation system of FIG. 1.

FIG. 2B is an enlarged view of a portion of the resection guide of FIG. 2A.

FIG. 2C is another enlarged view of a portion the resection guide of FIG. 2A.

FIG. 3A is a perspective view of a flex plate of the bone preparation system of FIG. 1.

FIG. 3B is a side view of the flex plate of FIG. 3A.

FIG. 3C is an enlarged view of a portion of the flex plate of FIG. 3A.

FIG. 3D is a perspective view of a flex plate according to one embodiment of the present disclosure.

FIG. 4A is a perspective view of a stem bushing of the bone preparation system of FIG. 1.

FIG. 4B is a perspective top view of the stem bushing of FIG. 4A.

FIG. 4C is a perspective bottom view of the stem bushing of FIG. 4A.

FIG. 4D is a side view of the stem bushing of FIG. 4A.

FIG. 4E is another side view of the stem bushing of FIG. 4A.

FIGS. 5-12 show steps in a method of using a bone preparation system according to an embodiment of the present disclosure.

### DETAILED DESCRIPTION

As used herein, the terms “about,” “generally,” and “substantially” are intended to mean that slight deviations from absolute are included within the scope of the term so modified. To aid the Patent Office and any readers of any patent issued on this application in interpreting the claims appended hereto, Applicant notes that it does not intend any of the appended claims or claim elements to invoke 35 U.S.C. § 112 (f) unless the words “means for” or “step for” are explicitly used in the particular claim.

As used herein unless stated otherwise, the term “anterior” means toward the front part of the body, and the term “posterior” means toward the back part of the body. When referring to specific directions in the following description, the terms “proximal” and “distal” are to be understood in regard to the device's orientation and position relative to an operator during exemplary application to the human body. Thus, the term “proximal” means closer to the operator or in a direction toward the operator, and the term “distal” means more distant from the operator or in a direction away from the operator.

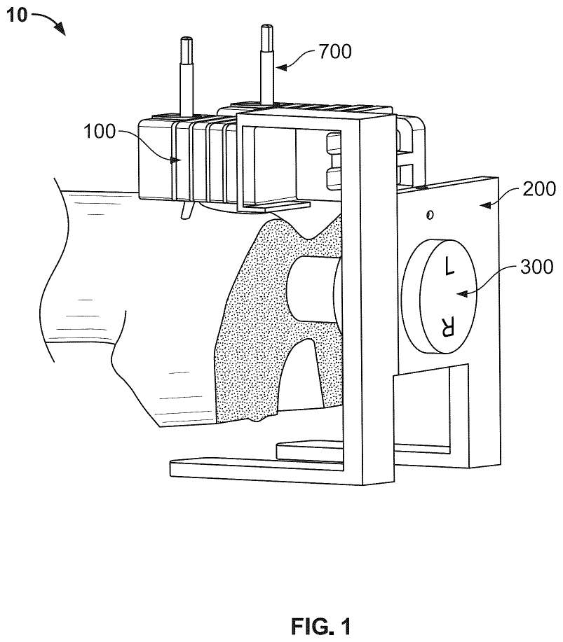

In one aspect, the present disclosure relates to an improved system for preparing a bone to receive an implant, and specifically for preparing a femur to receive a femoral implant during an initial or revision total knee arthroplasty (TKA) procedure. FIGS. 1-12 illustrate a bone preparation system 10 according to one embodiment. In one example, system 10 is used for determining and preparing a planned resection of a portion of the distal femur to receive a femoral implant. System 10 is used in a revision TKA procedure to allow a user to control the amount of flexion in the knee based on controlled resection of the distal femur, as well as allowing the user to determine and minimize a flexion gap of the knee. Further, system 10 allows a user to determine whether the angle of resection does not prevent the user from implanting a stem component within the IM canal of the distal femur. While system 10 is depicted and described for use in a femur of a patient, system 1 may be used in other long bones such as the tibia and fibula. System 10 includes a resection guide 100, a flex plate 200, and a stem bushing 300. In some embodiments, system 10 also includes one or more bone fixation pins 700, an intramedullary (IM) canal reamer 400, a resection guide tower 500, and/or a support arm assembly 600. Resection guide 100, flex plate 200, stem bushing 300, and pins 700 may be comprised of titanium alloy, cobalt chrome, or any other commonly used metal alloys, such as stainless steel. We now turn to the various components of the system. It should be appreciated that individual components of the system are also contemplated as individual standalone devices.

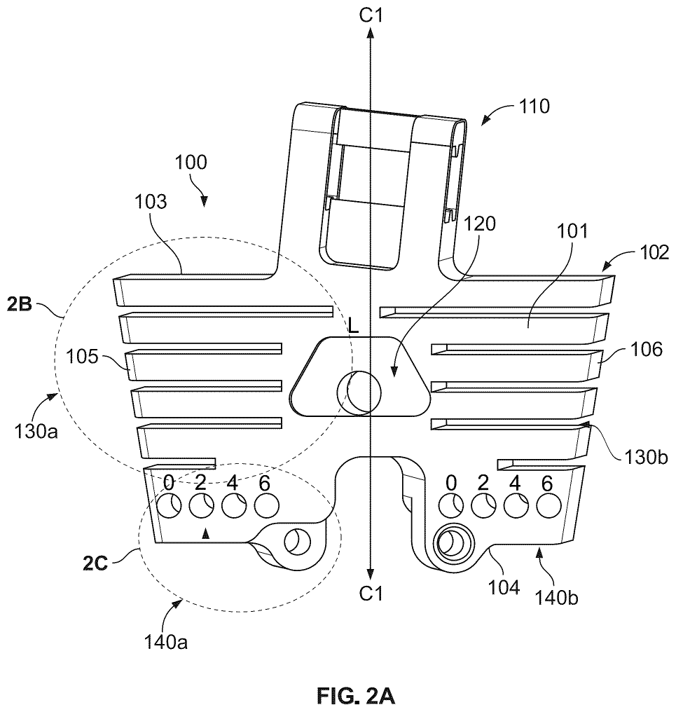

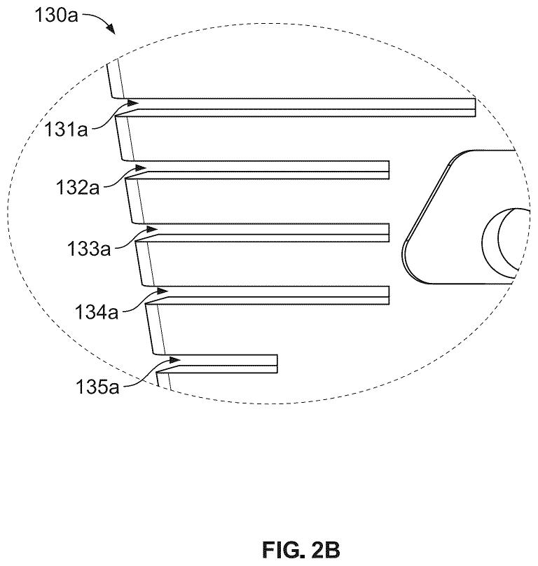

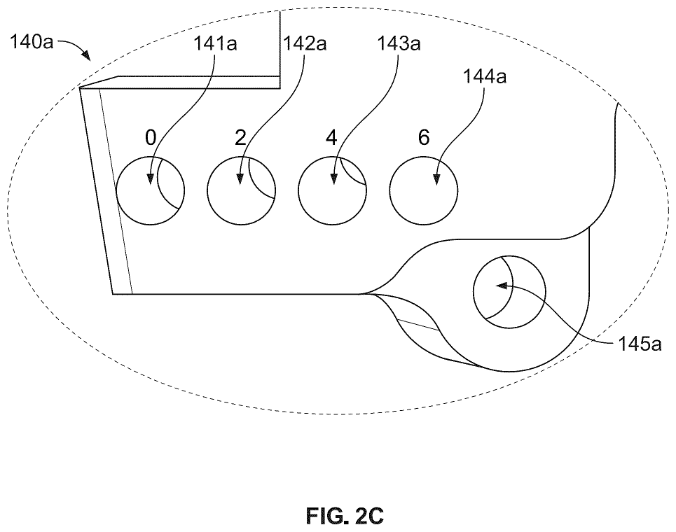

Resection guide 100 of system 10 is shown in FIG. 2A-2C. Resection guide 100 includes a support arm attachment feature 110, an alignment handle attachment feature 120, a plurality of slots 130, and a plurality of pin holes 140. As discussed further below, resection guide 100 is designed to be anchored to a distal portion of a femur during a revision TKA procedure.

Support arm attachment feature 110 extends from a first end 103 of resection guide 100 and includes a plurality of openings to receive a portion of a support arm assembly 600, as discussed further below. Alignment handle attachment feature 120 includes an opening that extends through a front or user facing surface 101 of resection guide 100 to a rear or bone-facing surface 102 of resection guide 100. Alignment handle attachment feature 120 is positioned approximately at a center position of resection guide 100 and may facilitate additional anchorage for resection guide 100 along with additional bone visualization.

Plurality of slots 130 extend through front surface 101 to rear surface 102 resection guide 100. Further, plurality of slots 130 includes a first group of slots 130a and a second group of slots 130b. First group of slots 130a extend from a first side surface 105 of resection guide 100 laterally towards, but are spaced apart from, a central longitudinal axis C1 extending through resection guide 100, and second group of slots 130b extend from a second side surface 106 of resection guide 100 also laterally towards, but are spaced apart from, the central longitudinal axis C1. In the depicted embodiment, first group of slots 130a are designed to match second group of slots 130b such that a respective slot from each grouping matches a respective slot from the other grouping. For example, respective slots from the first and second group of slots 130a, 130b may be designed to have the same dimensions, such as the width, height, and length through the resection guide 100. Therefore, the discussion below for first group of slots 130a should also be construed as describing the second group of slots 130b. Although not explicitly described, it should be appreciated that variations may have one or more differences between the first and second groups of slots.

An enlarged view of the first group of slots 130a is shown in FIG. 2B. First group of slots 130a includes a first slot 131a, a second slot 132a, a third slot 133a, a fourth slot 134a, and a fifth slot 135a. However, while first, second, third, fourth, and fifth slots 131a, 132a, 133a, 134a, 135a are shown and described below, resection guide 100 may include only one of these slots or may include additional slots. First slot 131a is near first end 103 of resection guide 100 and fifth slot 135a is near a second end 104 of resection guide 100. First slot 131a extends furthest laterally through resection guide 100 to an end proximate the central longitudinal axis C1 extending through resection guide. Fifth slot 135a extends shortest laterally through resection guide 100, approximately half the distance of first slot 131a. Fifth slot 135a is also referred to as the Medial Epicondyle (ME) slot when used in system 10 and attached onto a portion of the distal femur, as discussed further below. Second, third, and fourth slots 132a, 133a, 134a each extend laterally through resection guide 100 a distance in between that of first slot 131a and fifth slot 135a. Each slot of the first group of slots 130a is configured to receive a cutting tool for resecting a portion of the distal femur, as discussed further below. In the depicted embodiment, in a direction along the central longitudinal axis C1 extending through the resection guide, first slot, second, third, and fourth slots 131a, 132a, 133a, 134a are evenly spaced apart by 5-millimeter increments. As discussed further below, this design allows for varying depths of resection for a portion of the distal femur when used with system 10. For example, slot 131a corresponds to an initial distal femur resection depth of 0 mm, slot 132a corresponds to a resection depth of 5 mm, slot 133a corresponds to a resection depth of 10 mm, and 134a corresponds to a depth of 15 mm. While a distance between each slot of the first group of slots 130a is shown as equal to 5 mm, this distance may be within a range of 1 mm to 10 mm. In variations, the length of each slot of the first group of slots 130a may vary from that shown, in absolute terms or relative to another slot. Additionally, the distance between each slot of the first group of slots 130a does not need to be equal and can vary between each respective slot.

Resection guide 100 further includes a plurality of pin holes 140 extending through front surface 101 to rear surface 102 of resection guide 100. Plurality of pin holes 140 are disposed adjacent to second end 104 of resection guide 100 and, in the depicted embodiment, are each configured to receive a cylindrical bone fixation pin 700 for anchoring resection guide 100 to a portion of the distal femur, as discussed further below. In variations, plurality of pin holes 140 may comprise different shapes corresponding to alternative designs or shapes of bone fixations pins 700. Plurality of pin holes 140 include a first group of pin holes 140a and a second group of pin holes 140b. First group of pin holes 140a are disposed through a first side of resection guide 100, the first side of resection guide being defined from the first side surface 105 to approximately the central longitudinal axis C1 of resection guide 100. Second group of pin holes 140b are disposed through a second side of resection guide 100, the second side of resection guide being defined from the second side surface 106 to approximately the central longitudinal axis C1 of resection guide 100. First group of pin holes 140a are designed to match second group of pin holes 140b such that a respective pin hole from the first grouping matches a respective pin hole from the second grouping. For example, a respective pin hole from the first and second group of pin holes 140a, 140b will have the same dimensions, such as the circumference, angle, width, and height through the resection guide 100. Therefore, the discussion below for first group of pin holes 140a should be understood as representative of the second group of pin holes 140b.

An enlarged view of the first group of pin holes 140a is shown in FIG. 2C. First group of pin holes 140a includes a first pin hole 141a, a second pin hole 142a, a third pin hole 143a, a fourth pin hole 144a, and a fifth pin hole 145a. However, while first, second, third, fourth, and fifth pins holes 141a, 142a, 143a, 144a, 145a are shown and described below, resection guide 100 may include any number of these pin holes, e.g., one two or three, or may include additional pin holes. A center of the openings of the first, second, third, and fourth pin holes 141a, 142a, 143a, 144a on the front surface 101 are substantially aligned along a lateral axis extending through a width of the resection guide 100, wherein first pin hole 141a is disposed through the resection guide 100 closest to first side surface 105, and fourth pin hole 144a is disposed through resection guide 100 closest to the central longitudinal axis C1. Fifth pin hole 145a is disposed through resection guide 100 below first, second, third, and fourth pin holes 141a, 142a, 143a, 144a such that fifth pin hole 145a is closest to second end 104 of resection guide. Each pin hole for the first group of pin holes 140a is designed to receive pin 700 for anchoring resection guide 100 to a portion of the distal femur.

With continued reference to pin holes 140a, a central axis or trajectory for each pin 700 extending through each pin hole for first group of pin holes 140a is angled relative to a plane extending through first slot 131a, as discussed further below. Specifically, the central axis extending through first pin hole 141a parallel to the plane extending through first slot 131a, the central axis extending through second pin hole 142a is angled two degrees relative to the plane extending through first slot 131a, the central axis extending through third pin hole 143a is angled four degrees relative to the plane extending through first slot 131a, and the central axis extending through fourth pin hole 144a is angled six degrees relative to the plane extending through first slot 131a. In the depicted embodiment, each angled pin hole for the first group of pin holes 140a is closer to the slot on front surface 101 of the resection guide 100 than on the rear surface 102. In variations, resection guide 100 may include more or fewer pin holes than shown in the depicted embodiment. Additionally, any of the pin holes may be designed to have differing central axes angled relative to the plane extending through first slot 131 than what is shown in the depicted embodiment. For example, any of the pin holes may be angled greater than six degrees relative to the plane extending through first slot 131, and any of the pin holes may be angled less than zero degree (i.e., a “negative” angle) relative to the plane extending through first slot 131. Further, any of the pin holes may be angled relative to the central longitudinal axis C1 extending through resection guide 100.

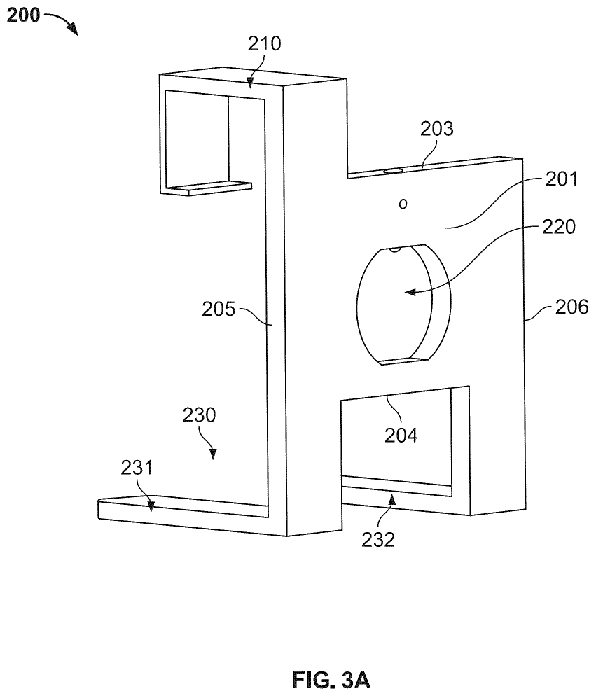

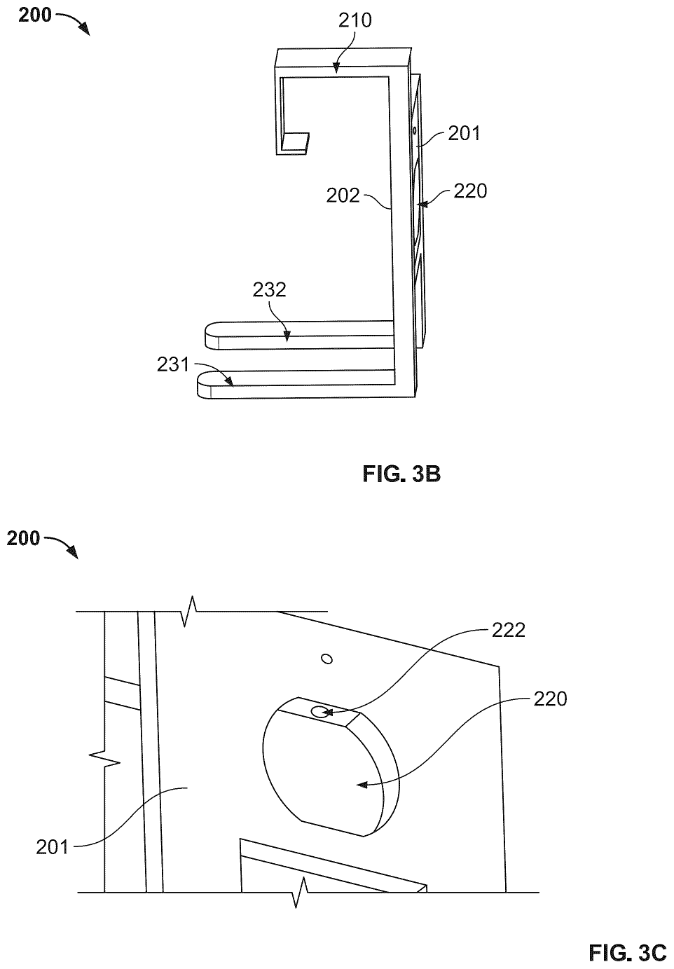

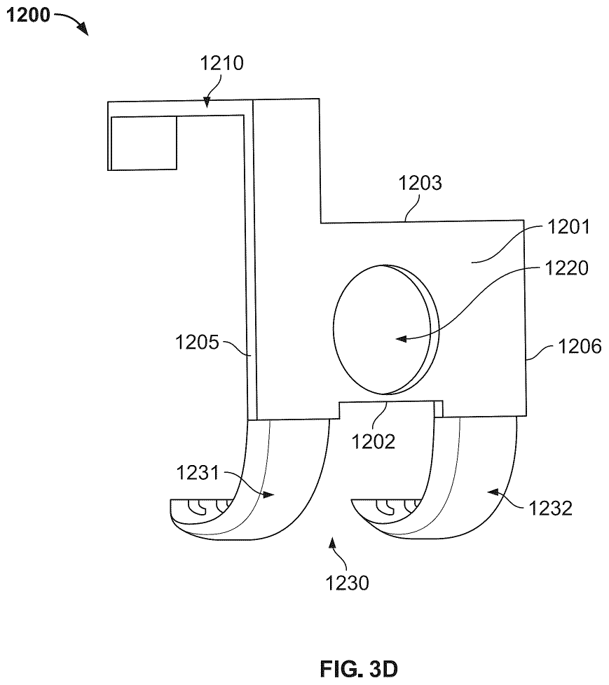

Flex plate 200 of system 10 is shown in FIG. 3A-3D. Flex plate 200 includes an engagement tab 210, a stem bushing opening 220, and a plurality of feet 230. Engagement tab 210 extends from a first end 203 of flex plate 200 in a direction opposite of a front surface 201. Additionally, engagement tab 210 extends from first end 203 adjacent a first side surface 205 of flex plate 200. As depicted, each surface of engagement tab 210 is substantially planar and connected in a perpendicular manner. As discussed further below, engagement tab 210 is designed to releasably engage any one of second group of slots 130b of resection guide for fixing flex plate 200 to resection guide 100. In other variations, the flex plate may be designed for engagement with first group of slots 130a.

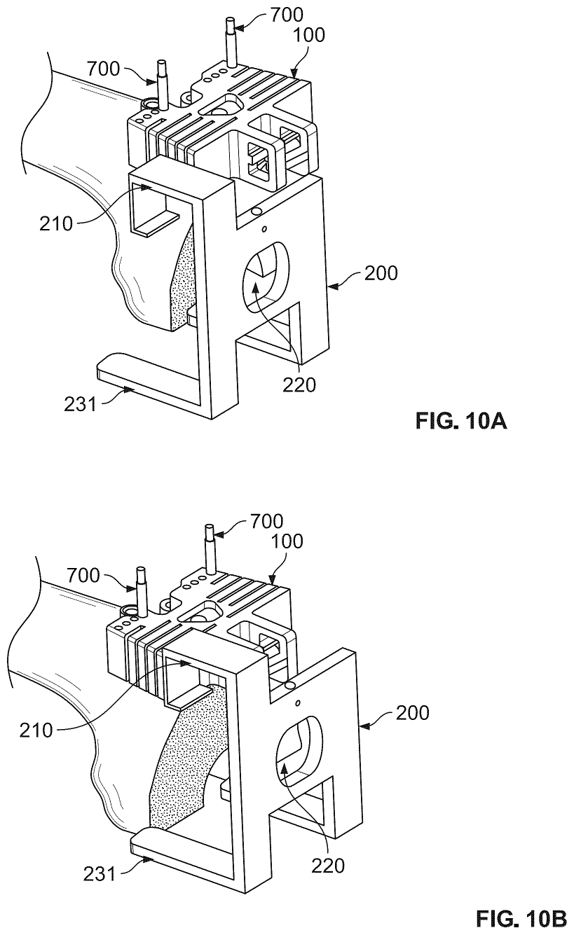

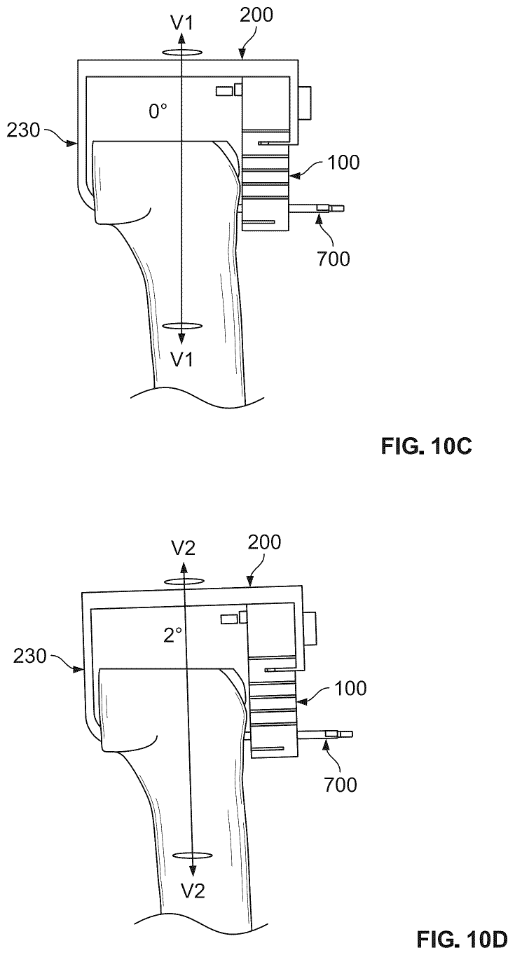

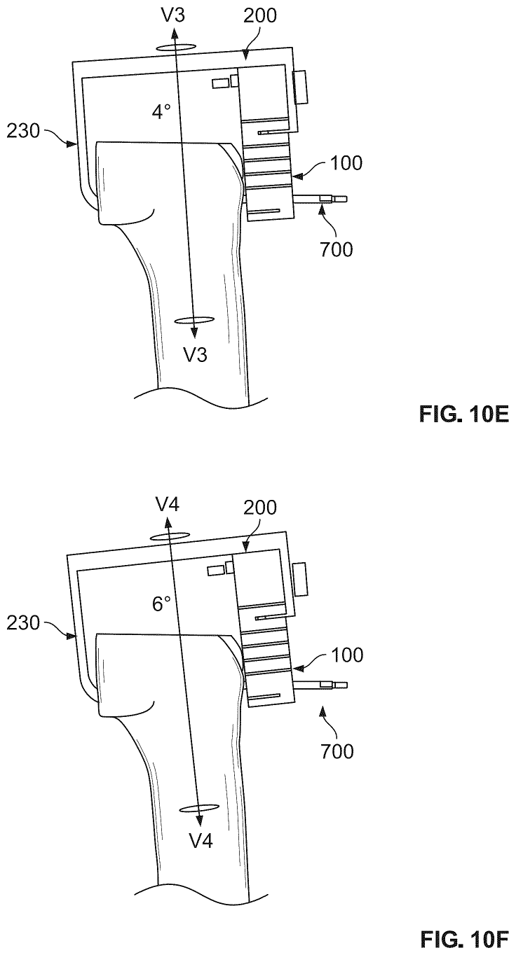

Stem bushing opening 220 extends through front surface 201 to a rear surface 202 of flex plate 200. Stem bushing opening 220 is substantially circular and is positioned at an approximately central point of flex plate 200. In variations, a shape of the stem bushing opening 220 may be non-circular as appropriate to accommodate a reaming tool, such as a boss reamer or IM reamer, and/or stem. Stem bushing opening 220 is designed to receive and align stem bushing 300 disposed partially within the intramedullary canal of the distal femur, as discussed further below. As depicted, an inner surface of stem bushing opening 220 between front surface 201 and rear surface 202 includes a ball detent 222. Ball detent 222 is designed to be received within either of ball detent openings 314, 315 of stem bushing 300, as discussed further below. In variations, flex plate 200 may have another engagement mechanism other than a ball detent to prevent rotation of stem bushing 300 while positioned through stem bushing opening 220. Prevention of rotation may also be achieved through a shaping of the stem bushing opening to include one or more flat surfaces, as shown in FIGS. 10A-B, for example. In still further variations, it may simply be an opening without any engagement mechanism.

Plurality of feet 230 extend from a second end 204 of base plate 200 opposite first end 203. Plurality of feet 230 may include a first foot 231 and a second foot 232 each extending from second end 204 of base plate 200 in a direction opposite front surface 201. First foot 231 extends from flex plate 200 adjacent first side surface 205 of flex plate 200, and second foot 232 extends from flex plate 200 adjacent second side surface 206 of flex plate 200. Each surface of the plurality of feet 230 is substantially planar and connected in a perpendicular manner. In one alternative embodiment, as shown in FIG. 3D, a flex plate 1200 differs from flex plate 200 only in the design of a plurality of feet 1230. In flex plate 1200, first foot 1231 and second foot 1232 each include a generally rounded, monolithic surface extending from second end 1202 in a direction opposite front surface 1201 of flex plate 1200. In each embodiment of flex plate 200, 1200, the respective plurality of feet 230, 1230 are designed to contact a portion of the distal femur when flex plate 200 is fixed to resection guide 100 and resection guide 100 is, in turn, attached to the distal femur, as discussed further below.

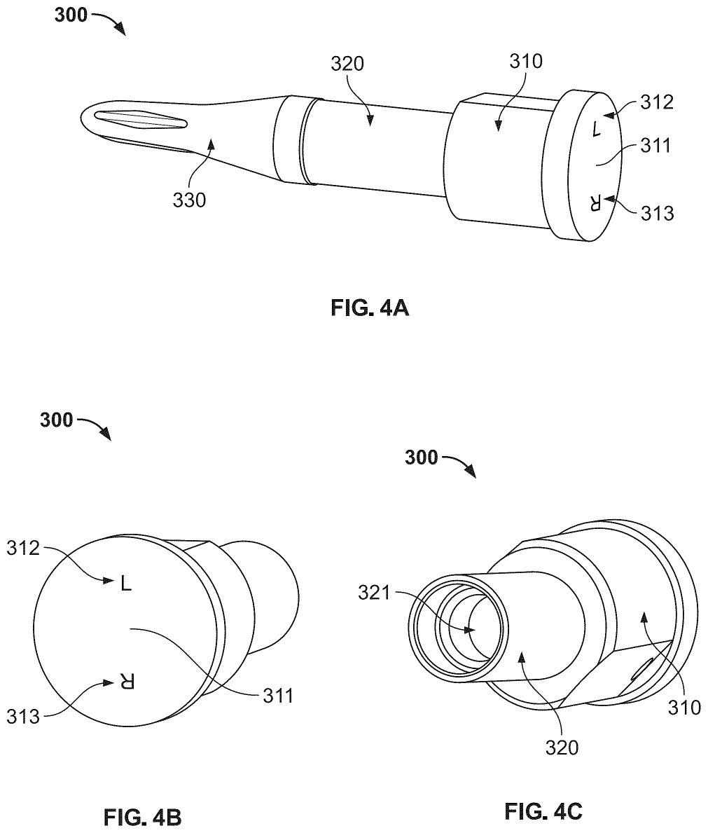

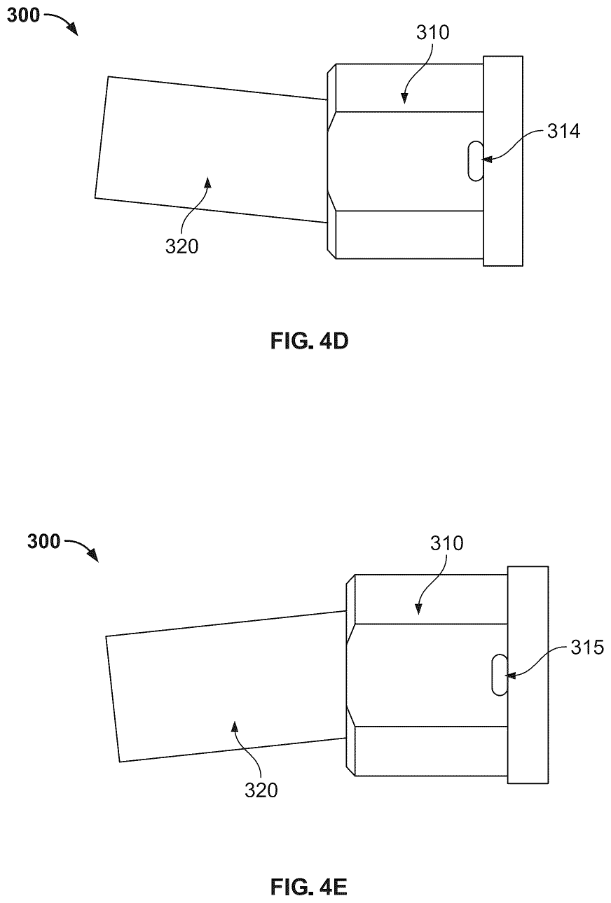

Stem bushing 300 of system 10 is shown in FIGS. 4A-4E. Stem bushing 300 includes a head portion 310 and a shaft portion 320 extending from the head portion 310. Head portion 310 is substantially cylindrical in shape and includes a planar top surface 311. As depicted, top surface 311 includes a left configuration marking 312 and right configuration marking 313 for determining the correct extension of shaft portion 320 and alignment of stem bushing in system 10 within either the left or right femur, as discussed further below. Head portion 310 includes a first ball detent opening 314 and a second ball detent opening 315 disposed on opposite sides of head portion 310. Each of the ball detent openings 314, 315 are designed to engage ball detent 222 of flex plate 200 in order to fixedly retain stem bushing 300 in flex plate 200. In variations, flex plate 200 may include the ball detent openings and the stem bushing 300 may include the ball detents.

Shaft portion 320 may extend away from head portion 310 at an angle to match one of either the left or right valgus angle of the distal femur, as best shown in FIGS. 4D-4E, depending upon the positioning of stem bushing 300 within stem bushing opening 220 of flex plate 200. A length of shaft portion 320 is designed to match a length of a boss of a femoral component implanted in the distal femur. A distal end of shaft portion 320 opposite head portion 310 includes a threaded hole 321 designed to connect with a portion of a stem trial 330.

In another aspect, the present application relates to a kit including a combination of components including at least one component of the system 10. In some embodiments, a kit may include one or more components from system 10. For example, the kit may include a resection guide 100, one or more flex plates 200, one or more stem bushings 300, one or more pins 700. In some examples, the kit may further include one or more of an intramedullary canal reamer 400, guide tower 500, support assembly 600, and a bone resecting instrument.

Any combination of system components may also be included in a single package or in individual packages which may later be brought together to create a kit. When more than one package is used, any one package in such kit may include one or more of the components of the kit. It is also contemplated that a kit may include any combination of system components along with one or more additional instruments used to place such securement devices in a patient. In other examples, the kits contemplated herein may be accompanied by an instruction manual on how to perform one or more of the methods of using the contents of the kit.

In another aspect, the present disclosure relates to a method for performing a surgical procedure for preparing a bone to receive an implant, and specifically for preparing a femur to receive a femoral implant during a revision TKA procedure. As explained elsewhere in the present application, it should be appreciated that the system and components thereof as contemplated by the present disclosure are not limited to use in revision TKA procedures. And, to the extent the methods below are described with respect to a revision TKA procedure, such description is for purposes of illustration. It should be understood that the following operations do not have to be performed in the exact order described below. Instead, various steps may be handled in a different order or simultaneously. Steps may also be omitted or added unless otherwise stated herein.

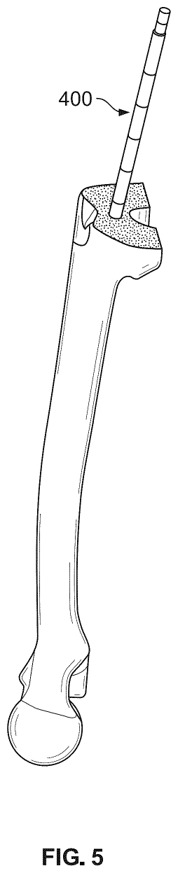

In one embodiment, a method for preparing a bone for receiving an implant begins with a first step of reaming the intramedullary canal of a distal femur. As previously discussed, an implant is configured for surgical procedures in either the left or right distal femur. In one non-limiting example, the intramedullary canal of a left femur is reamed by an IM reamer 400, as best shown in FIG. 5. For the sake of brevity, the description below focuses on a procedure on the left femur, but it should be appreciated that the contemplated method steps may be similarly employed in a right femur. The intramedullary canal of the femur is progressively reamed until cortical chatter is achieved, and then the IM reamer 400 should be firmly seated within the intramedullary canal.

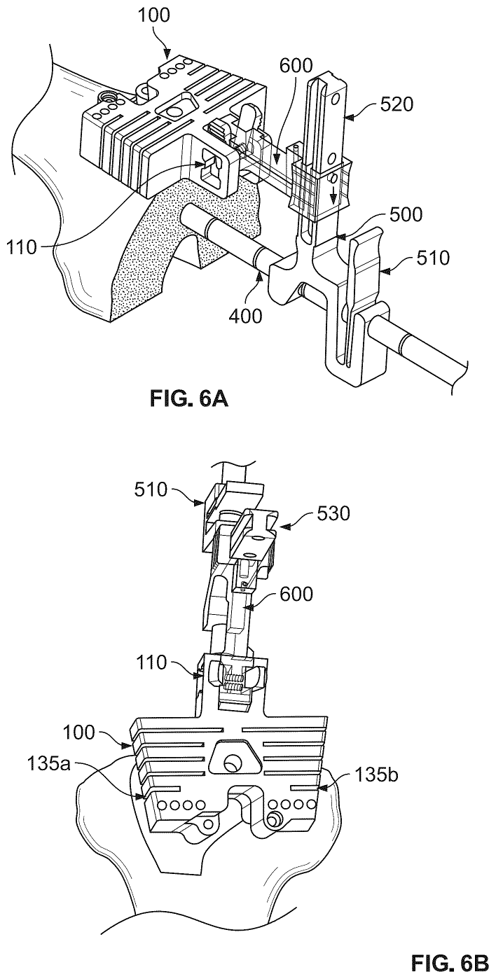

In a second step, resection guide tower 500 is connected the seated IM reamer 400 by depressing a finger tab 510 of resection guide tower 500 while sliding resection guide tower 500 over IM reamer 400, as best shown in FIGS. 6A-6B. One end of support arm assembly 600 is connected to support arm attachment feature 110 of resection guide 100 and an opposite end of support arm assembly 600 is connected to resection guide tower attachment feature 520.

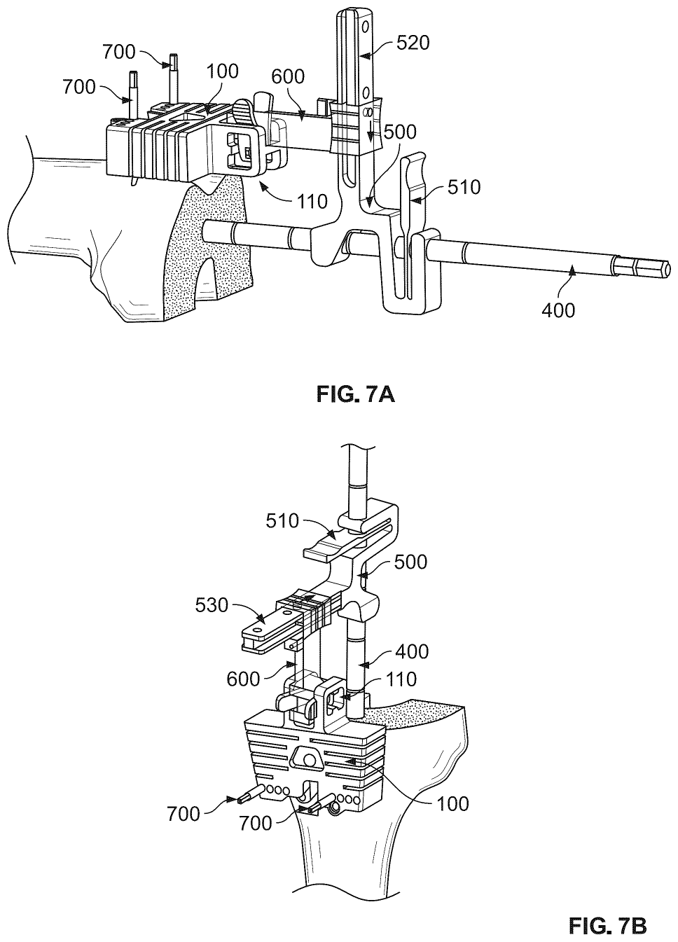

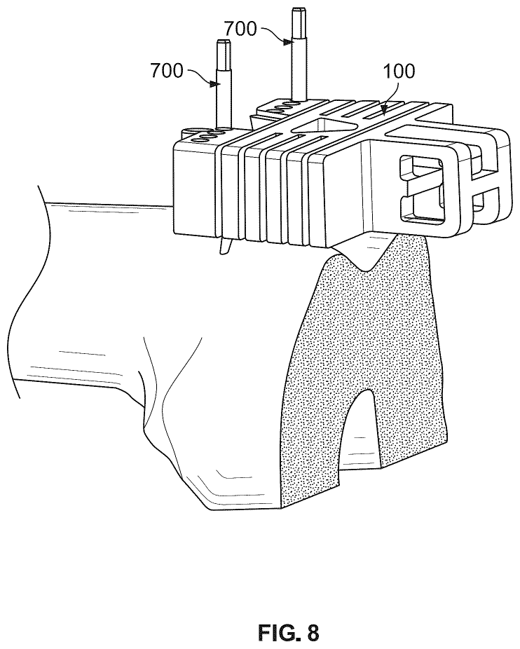

In a third step, the fifth slot or ME slot 135a, 135b of the resection guide is positioned to be aligned with the medial epicondyle of a portion of the distal femur. After satisfactory alignment has been determined, first and second pins 700 are anchored into a portion of the distal femur through respective first pin holes 141a, 141b of the first and second groups of pin holes 140a, 140b of resection guide 100, as best shown in FIGS. 7A-7B. In this manner, the anchored pins 700 allow for resection guide 100 to be attached to a portion of the distal femur.

In a fourth step, support arm assembly 600 is disconnected from support arm attachment feature 110 of resection guide 100, and guide tower 500, support arm assembly 600, and IM reamer are removed from the femur, as best shown in FIG. 8. Thus, at this step, only resection guide 100 remains selectively anchored to a portion of the distal femur, such anchorage being via the connection with each of pins 700 through respective first pin holes 141a, 141b.

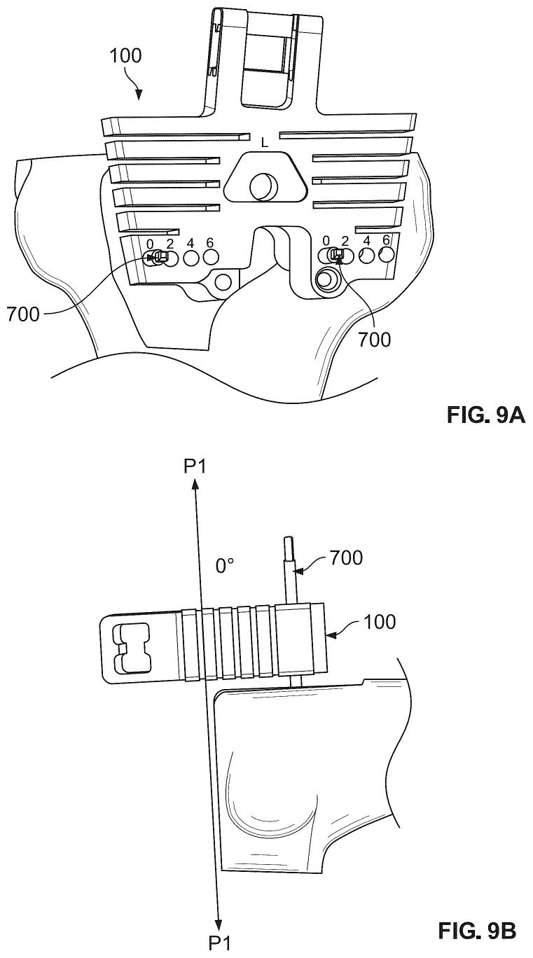

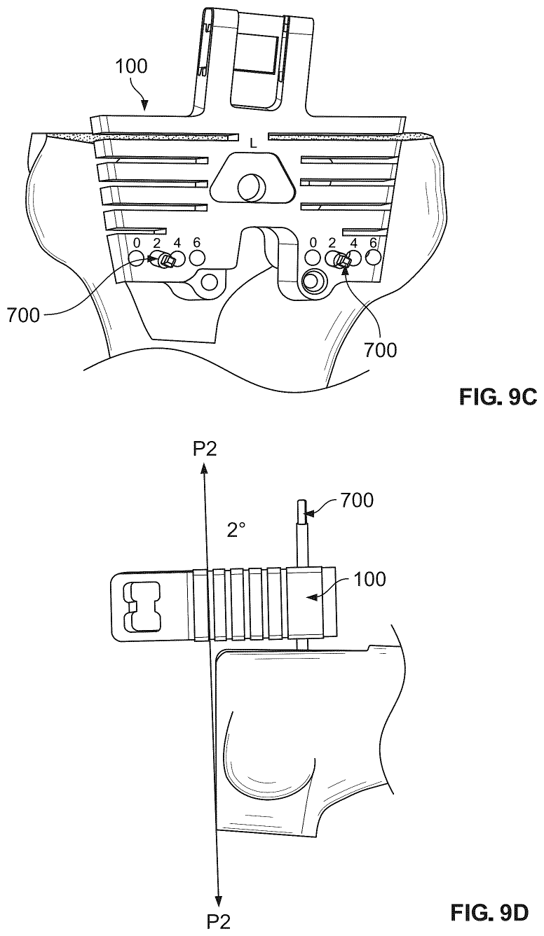

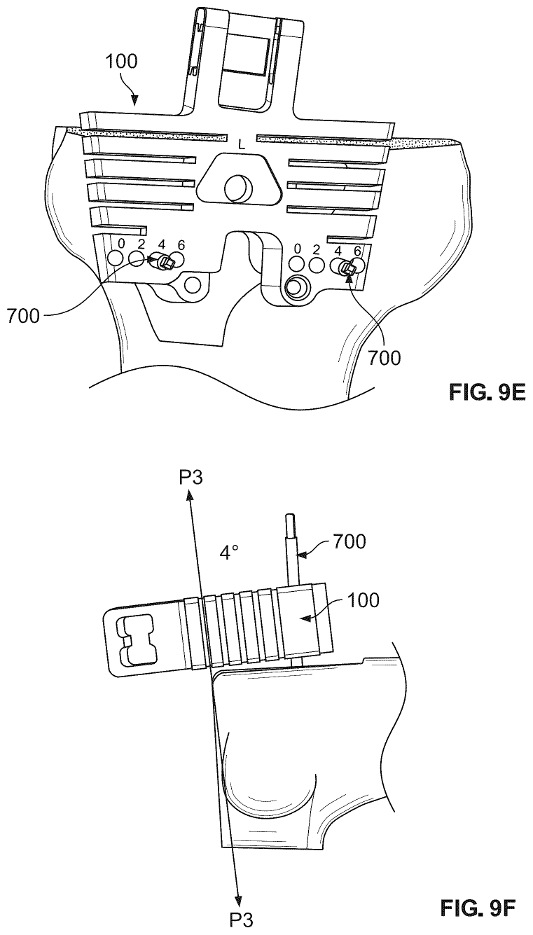

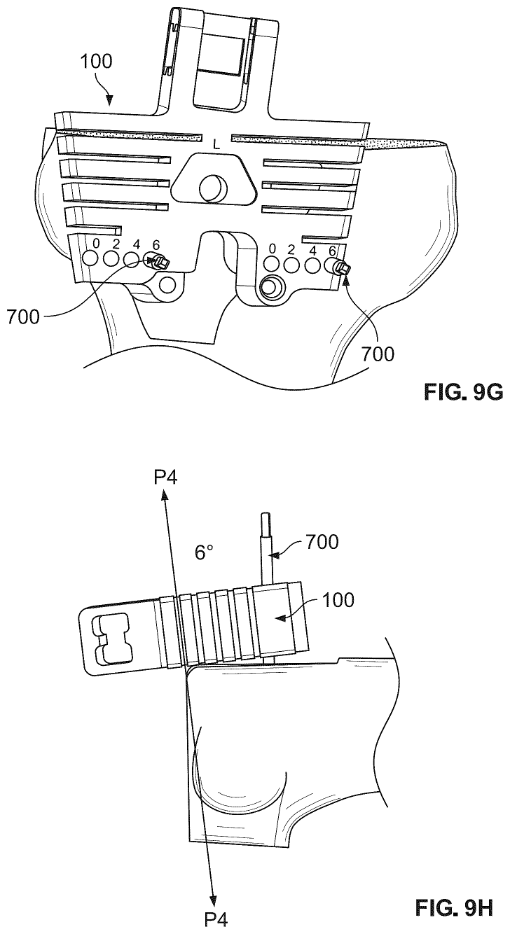

In a fifth step, resection guide 100 is selectively repositioned over pins 700 in order create a desired flexion or resection angle, i.e., a desired resection cut line on a distal end surface of the distal femur, as best shown in FIGS. 9A-9H. The desired flexion or resection angle may be described as an angle relative to a plane that is normal to a central longitudinal axis of the femur. For example, as shown in FIGS. 9A-9B, when resection guide 100 is in a first position over each pin 700 respectively through first pin holes 141a, 141b, first slots 131a, 131b are positioned along a first plane P1 extending through a portion of the distal femur in the anterior/posterior direction. The first position may also be referred to as an “initial position” of resection guide 100, and the first plane P1 may be considered to have a resection angle of zero degrees because first plane P1 is normal to the central longitudinal axis of the femur.

At this stage of the method, a surgical plan or otherwise any real time considerations of a patient's anatomy may call for continuing with bone preparation based on the zero-degree resection angle or may involve consideration of other resection cut angles, which resection guide 100 may accommodate. If the procedure requires a different resection angle and/or positioning of the guide to set up stem placement, then step six is performed.

In the sixth step, resection guide 100 is slidably removed from pins 700, and then is repositioned over each pin 700 respectively through any pair of second pin holes 142a, 142b, third pin holes 143a, 143b, and fourth pin holes 143a, 143b. Resection guide 100 may be disposed over each pin 700 respectively through only one pair of pin holes, or this step may be repeated and the resection guide 100 is disposed over each pin 700 through another pair of pin holes. As shown in FIGS. 9C-9D, resection guide 100 is in a second position such that respective pins 700 are received through second pin holes 142a, 142b, thereby positioning first slots 131a, 131b along a second plane P2 extending through a portion of the distal femur in the anterior/posterior direction. Second plane P2 is angled relative to first plane by two degrees in a direction such that from the anterior side of the femur toward the posterior side of the femur, the plane extends in a slightly superior direction, as shown in FIG. 9D. As shown in FIGS. 9E-9F, resection guide 100 is in a third position such that respective pins 700 are received through third pin holes 143a, 143b, thereby positioning first slots 131a, 131b along a third plane P3 extending through a portion of the distal femur in the anterior/posterior direction. Third plane P3 is angled relative to first plane by four degrees in a direction such that from the anterior side of the femur toward the posterior side of the femur, the plane extends in a slightly superior direction, as shown in FIG. 9F. As shown in FIGS. 9G-9H, resection guide 100 is in a fourth position such that respective pins 700 are received through fourth pin holes 144a, 144b, thereby positioning first slots 131a, 131b along a fourth plane P4 extending through a portion of the distal femur in the anterior/posterior direction. Fourth plane P4 is angled relative to first plane by six degrees in a direction such that from the anterior side of the femur toward the posterior side of the femur, the plane extends in a slightly superior direction, as shown in FIG. 9H. Additionally, each pair of pin holes may optionally include a marking directly above the respective pin hole indicating the respective flexion angle, such as “0” over the first pin holes 141a, 141b, “2” over the second pin holes 142a, 142b, “4” over the third pin holes 143a, 143b, and “6” over the fourth pin holes 144a, 144b.

In a seventh step, flex plate 200 is connected to resection guide 100, as shown in FIGS. 10A-10E. Engagement tab 210 of flex plate 200 is slidably positioned within first slot 131b of the second group of slots 130b, However, engagement tab 210 may be slidably positioned within any slot of the first and second group of slots. Once engagement tab 210 is fully seated within first slot 131b, stem bushing opening 220 is positioned to align with a planned trajectory of a femoral stem component along a valgus angle through a portion of the distal femur. The planned trajectory and valgus angle through a central point of stem bushing opening 220 is dependent upon the position of resection guide 100, as discussed in the fifth and sixth steps above. For example, as shown in FIG. 10C, resection guide 100 is in the first or “initial” position with a first valgus angle V1 extending through the central point of stem bushing opening 220 and through a portion of the distal femur. As shown in FIG. 10D, resection guide 100 is in the second position with a second valgus angle V2 extending through the central point of stem bushing opening 220 and through a portion of the distal femur. Second valgus angle V2 is angled relative to the first valgus angle V1 by two degrees in an anterior/posterior direction along the distal femur. As shown in FIG. 10E, resection guide 100 is in the third position with a third valgus angle V3 extending through the central point of stem bushing opening 220 and through a portion of the distal femur. Third valgus angle V3 is angled relative to the first valgus angle V1 by four degrees in an anterior/posterior direction along the distal femur. As shown in FIG. 10F, resection guide 100 is in the fourth position with a fourth valgus angle V4 extending through the central point of stem bushing opening 220 and through a portion of the distal femur. Fourth valgus angle V4 is angled relative to the first valgus angle V1 by six degrees in an anterior/posterior direction along the distal femur. Additionally, in each position of the resection guide 100, feet 230 of flex plate 200 may engage a portion of the posterior femur, which designates a predicted posterior position of the femoral implant to be implanted. This allows the resection guide 100 to identify the predicted flexion gap prior to resecting the distal femur.

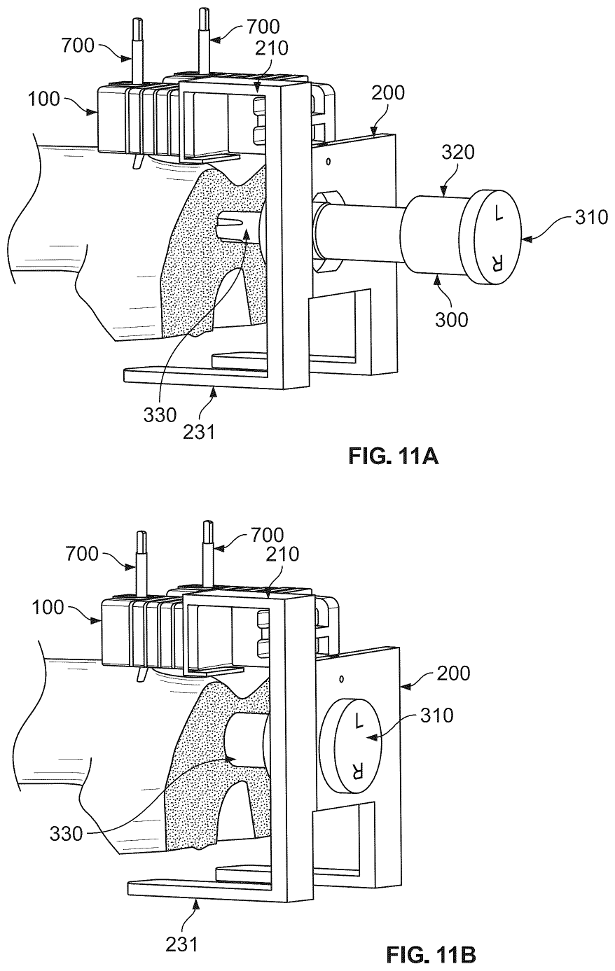

In an eighth step, stem bushing 300 is positioned through stem bushing opening 220 of flex plate 200 and into the reamed IM canal of the distal femur, as best shown in FIGS. 11A-11B. Stem bushing 300, in connection with flex plate 200, allows a user to determine if a stemmed femoral component is able to be implanted at this flexion and resection angle, and stem bushing 300 represents the valgus angle of the stemmed femoral component to be implanted into the femur. Specifically, stem bushing opening 220 provides varying alignments for the stem bushing 300 to be received within the reamed IM canal. In the depicted embodiment, stem bushing 300 is slightly angled relative to the alignment of the stem bushing opening 220 into the reamed IM canal. Therefore, when stem bushing 300 is received within stem bushing opening 220, the stem bushing 300 is angled when positioned within the reamed IM canal. In variations, stem bushing 300 may be entirely linear without being angled, and in these embodiments, stem bushing 300 will not be positioned along an angle within the reamed IM canal.

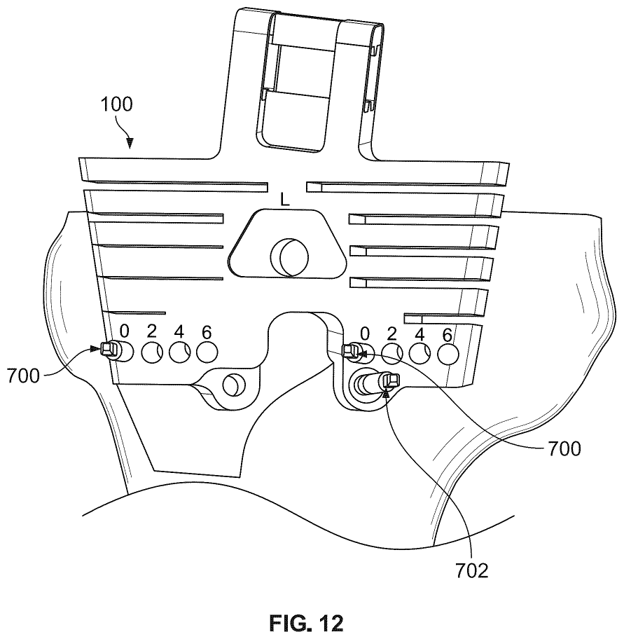

In a ninth step, a user confirms the desired resection angle and removes stem bushing 300 and flex plate 200 from resection guide 100. In the depicted embodiment in FIG. 12, the desired resection angle through first slots 131a, 131b of resection guide 100 is along first plane P1 extending through a portion of the distal femur in the anterior/posterior direction. Therefore, resection guide 100 is in the first position over each pin 700 respectively through first pin holes 141a, 141b. Resection guide 100 is then finally positioned by anchoring an additional pin 702 through fifth pin hole 145b and into a portion of the distal femur, as best shown in FIG. 12. While the depicted embodiment shows the resection guide 100 finally positioned to the distal femur through fifth pin hole 145b, resection guide 100 may be anchored by an additional pin 702 extending through fifth pin 145a, or resection guide 100 may only be anchored by an additional pin 702 extending through fifth pin holes 145a. Pin holes 145a, 145b extend through resection guide 100 in a medial lateral direction through the distal femur. Therefore, anchoring resection guide 100 to a portion of the distal femur through at least one of pin holes 145a, 145b prevents movement or backing out of resection guide 100 during any vibrations that may occur during resections made through resection guide 100. Thus, once resection guide 100 is in the final position, resections through any of the respective slots may be made to prepare the distal femur for receiving a femoral implant.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cutting guide for resecting a bone to receive an implant, the cutting guide comprising:
a body configured to be positioned adjacent to the bone;
a slot extending through the body and configured to receive a cutting edge for resecting the bone;
a first hole extending though the body and configured to receive a pin anchored to the bone, the first hole being configured to position the slot along a first plane when the first hole is passed over the pin; and
a second hole extending though the body and configured to receive the pin anchored to the bone, the second hole being configured to position the slot along a second plane when the second hole is passed over the pin,
wherein the first plane and the second plane are both orthogonal to a third plane, and the first plane is transverse to the second plane, and
wherein the cutting guide is configured such that when the body is received on the pin anchored to the bone, the first plane represents a first path of resection relative to the bone and the second plane represents a second path of resection relative to the bone.

2. The cutting guide of claim 1, wherein the body is configured to be positioned adjacent an anterior side of the bone such that the cutting edge resects the bone in an anterior-posterior direction.

3. The cutting guide of claim 1, wherein an angle between the first plane and the second plane is one of 2 degrees, 4 degrees or 6 degrees.

4. The cutting guide of claim 1, further comprising a third hole and a fourth hole, the third hole extending through the body and being configured to receive a second pin anchored to the bone when the first hole receives the pin, and the fourth hole extending through the body and being configured to receive the second pin anchored to the bone when the second hole receives the pin.

5. The cutting guide of claim 4, wherein a longitudinal axis divides the body into a first and second side, the first hole is adjacent to the second hole on the first side, and the third hole is adjacent to the fourth hole on the second side.

6. The cutting guide of claim 1, wherein the second hole is non-parallel to the first hole.

7. The cutting guide of claim 1, wherein the slot is proximate a first end of the body and the first and second holes are proximate a second end of the body opposite the first end, wherein the first end of the body is configured to be proximate a first end of bone.

8. The cutting guide of claim 1, further comprising a support arm assembly configured to connect the cutting guide to a reamer, wherein the reamer is configured to engage an intramedullary canal of the bone.

9. The cutting guide of claim 8, wherein the support arm assembly is further configured to position the slot along the first plane.

10. The cutting guide of claim 1, further comprising a plate configured to be received by the slot, wherein the plate comprises an opening configured to receive a stem along the third plane.

11. The cutting guide of claim 1, further comprising a second slot extending through the body, wherein the second slot is positioned one of 5 millimeters, 10 millimeters, or 15 millimeters from the slot.

12. A system for preparing a bone for receiving an implant, the system comprising:
a cutting guide configured to be attached to the bone, the cutting guide including a cutting slot and a plurality of holes, each hole of the plurality of holes being sized to receive a bone anchorage pin, wherein a first hole of the plurality of holes is configured to be received over the bone anchorage pin anchored to the bone so that the cutting guide is at a first orientation relative to the bone, and a second hole of the plurality of holes is configured to be received over the bone anchorage pin anchored to the bone so that the cutting guide is at a second orientation relative to the bone; and
a plate releasably engaged to the slot of the cutting guide, the plate including an arm extending from the slot and a guide portion parallel to the slot, the guide portion including an opening therethrough,
wherein when the plate is engaged to the slot and the cutting guide is attached to the bone in the first orientation, the opening is aligned along a first central longitudinal axis and when the plate is engaged to the slot and the cutting guide is attached to the bone in the second orientation, the opening is aligned along a second central longitudinal axis, the first central longitudinal axis having a different anterior-posterior component than the second central longitudinal axis.

13. The system of claim 12, wherein the bone anchorage pin is fixed to the bone along a first axis extending along an anterior-posterior direction of the bone.

14. The system of claim 12, further comprising a stem configured to engage the bone through the opening of the plate, wherein the bone is a left or right femur.

15. The system of claim 14, wherein the opening of the guide portion includes a detent for retaining a portion of the stem.

16. The system of claim 12, wherein an angle between the first central longitudinal axis and the second central longitudinal axis is one of 2 degrees, 4 degrees or 6 degrees.

17. The system of claim 12, wherein each hole of the plurality of holes of the cutting guide are further configured to receive a second bone anchorage pin, wherein a third hole of the plurality of holes is configured to be received over the second bone anchorage pin anchored to the bone when the first hole is configured to be received over the bone anchorage pin anchored to the bone and a fourth hole of the plurality of holes is configured to be received over the second bone anchorage pin anchored to the bone when the second hole is configured to be received over the bone anchorage pin anchored to the bone.

18. A method for preparing a bone for receiving an implant, the method comprising:
positioning a cutting guide along a portion of the bone;
anchoring a pin to the portion of the bone by passing the pin through a first hole in the cutting guide to align the cutting guide in a first orientation relative to the bone;
removing the cutting guide from the pin; and
advancing a second hole in the cutting guide over the pin anchored to the bone such that when the second hole receives the pin, the cutting guide is in a second orientation relative to the bone,
wherein in the first orientation, a first plane passes through a slot of the cutting guide, and in the second orientation, a second plane passes through the slot of the cutting guide, and
wherein the first plane defines a cut line through the bone at a different slope in an anterior-posterior direction than the second plane.

19. The method of claim 18, further comprising, prior to the positioning step, reaming an opening through the bone, wherein the opening is an intramedullary canal and the bone is a femur.

20. The method of claim 18, further comprising, after the advancing step, engaging a plate with the cutting guide, wherein the plate is configured to receive a stem extending through an opening of the plate into an opening of the bone to define a trajectory of the stem.
